(19) United States
(12) Patent Application Publication
(10) Pub. No.: US 2006/0144401 A1
(43) Pub. Date: Jul. 6, 2006

(54) Pressure reducing device

(76) Inventor: Poul Boelt, Jyllinge (DK)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/528,411

(22) PCT Filed: Sep. 18, 2003
(86) PCT No.: PCT/DK03/00605

§ 371 Date: Nov. 10, 2005

(30) Foreign Application Priority Data
Sep. 18, 2002 (PA) 2002-01372

(51) Int. Cl. A62B 9/02; A62B 7/10; A61M 15/08

(52) U.S. Cl. 128/205.24; 128/206.11; 128/207.18

(57) ABSTRACT

A pressure reducing valve for nasal supplying of a flow of air to a patient. The valve comprises a hollowed tubular member (1) comprising a high pressure air inlet end part (3), a low pressure air outlet end part (4) opposite to said inlet end part (3), and an intermediate air venting part having perforations (6, 7) for venting a constant and non-adjustable pressure and flow of air from the inner cavity of the tubular member (1) into the ambient atmosphere. A shielding member (FIG. 3) positioned above said perforations (6, 7) secures the perforations (6, 7) from becoming obstructed and for directing the air flow from said perforations (6, 7) towards said air inlet end part (3).

### FIELD OF THE INVENTION

The present invention relates to a pressure-reducing valve for installation in connection with an air/gas-conveying conduit. In general, the valve according to the present invention is adapted to significantly reduce the conveyed gas pressure by splitting a supplied stream of gas into more streams of gas. In particular, the invention relates to a pressure-reducing valve for nasal continuous positive airway pressure (CPAP) treatment on premature babies.

### BACKGROUND OF THE INVENTION

CPAP is an airway treatment wherein a slight positive air pressure is applied to the respiratory organs of a patient in order to increase the volume of inhaled air and thus, to decrease the work of breathing. CPAP treatment is given through a set of nasal prongs, through a mask or through a ventilation tube fixed in the trachea. Often, neonates, in particular premature babies, are given CPAP treatment in order to relieve the respiratory organs. Moreover, CPAP treatment is applied for the alleviation of snoring and obstructive sleep apnea. The applied air pressure should typically not exceed the equivalence of a 2-7 cm water column and the applied amount of air should not exceed the range of 5-7 litres per minute. Standard gas supplies are normally adapted to deliver gas of a much higher pressure and quantity and therefore a reduction valve is inserted between the gas supply and the CPAP treating device.

In general, valves for reducing air pressure and delivered air quantities, e.g. in connection with CPAP treatment, exist. Typically, a supplied stream of air, e.g. air supplied with 6-7 bar overpressure, is split into more streams of air. One of the streams is guided through one outlet to respiratory means, e.g. a set of nasal prongs or to a mask, and thus to the respiratory organs of the treated individual. The excess stream or streams of air is/are guided through other outlets to the ambient atmosphere. By splitting the 6-7 bar over-pressurised stream of air into several streams of air, the pressure and quantity of air guided to the respiratory means can be reduced significantly compared with the supplied air pressure. Due to its simple and yet highly reliable structure, this type of pressure reducing valve is appreciated not least for medical purposes such as CPAP treatment. It is however often experienced that the excess stream or streams of air emitted from the valve may cause an uncomfortable cooling and drying effect for the treated individual and during longer treatment cycles, complications deriving from the cooling and drying may occur.

It is crucial to secure a constant pressure and flow of air which cannot “by accident” be adjusted as it may have serious consequences for the patient, in particular when treating premature babies. Known valves comprise the feature of allowing the flow to be adjusted on the valve itself, and as the valve often need to be positioned in close vicinity of the baby, this feature makes them unsuitable for use in CPAP-treatment of particular premature babies, as there is a risk of adjustment “by accident”, e.g. if the baby touches the valve.

In the PCT publication WO 01/76658 there is disclosed a system for delivering warm, humidified oxygen to a patient at low flow rate. The system including a conduit having an inlet and an outlet, between the inlet and the outlet a flowpath extends through the wall for letting oxygen out. A member is movably positioned over the flowpaths for providing means for adjusting the flow of oxygen to the patient.

Other known devices for use within the respiratory field are disclosed in U.S. Pat. No. 5,937,851 A, U.S. Pat. No. 6,112,745 A, and DE 10121959 A. However these documents relate to the exhaust of CO₂ rather than for providing air or gas to a patient, and a fourth document, U.S. Pat. No. 5,042,478, relates to a nasal adaptor device.

### DESCRIPTION OF THE INVENTION

It is an object of the present invention to overcome the above-described disadvantages of the known pressure-reducing valves.

According to a first aspect, the present invention relates to a pressure reducing valve for nasal supplying of a flow of air to a patient, the valve comprising a hollowed tubular member comprising; a high pressure air inlet end part, a low pressure air outlet end part opposite to said inlet end part, an intermediate air venting part having perforation(s) for venting a constant and non-adjustable pressure and flow of air from the inner cavity of the tubular member into the ambient atmosphere, and wherein a shielding member is positioned above said perforation(s) for securing the perforation(s) from becoming obstructed and for directing the air flow from said perforation(s) towards said air inlet end part.

The pressure and flow of air is constant and cannot be adjusted on the valve itself for safety reasons, as it is i.e. very important to keep a constant overpressure inside the lunges of the patient. The pressure and flow may of course be adjusted prior to entering the valve, but preferably not at a location where there is a possibility of adjustment “by accident” which may have serious consequences for the patient.

Due to the arrangement of the perforations, an excess stream of air, i.e. a stream of air passing through the air venting part, is guided in a direction opposite the direction of the stream of air which is used for the treatment, i.e. the stream which is guided to the respiratory tract. Accordingly, the valve supports an arrangement wherein one stream of air is guided in one direction e.g. to a set of nasal prongs and other streams of air is blown in a direction facing away from this direction.

Upon suitable fitting of the valve with respect to nasal prongs etc., the air from the air venting part is blown in a direction facing away from the individual receiving the treatment. The valve is, at one end, connected to the gas-conveying conduit supplying pressurised gas. From this end, the supplied gas is split into a first flow of gas conveyed to the low pressure outlet and a second flow of gas conveyed to the ambient atmosphere, the two gas-flows being directed in opposite directions, or, at least the second flow of gas is directed in the direction towards the high pressure air inlet.

The valve is preferably provided in the form of an annular or tubular member defining a conduit between the high pressure air inlet and a low pressure air outlet. Between the high pressure air inlet and the low pressure air outlet, one or more air venting parts, i.e. parts for venting air to the ambient atmosphere may be defined. The air venting part or parts may be provided in the form of perforations of the wall of the tubular body, e.g. in the form of one or more air passages, such as through-going holes, i.e. holes extending from the conduit to an exterior surface of the annular or tubular member. On the exterior surface of the member, the air/gas flowing through the air venting part(s) is guided in a direction towards the high pressure air inlet. The through-going holes, i.e. the air venting part may extend radially from the conduit and may be provided, e.g. by drilling one or more holes in the side wall of the tubular member.

As an example, the hole(s) may be drilled throughout the side wall so that a first part of the supplied gas can flow through the conduit from the high pressure air inlet to the low pressure air outlet, e.g. to nasal prongs while a second part of the supplied gas can flow through the through-going holes to the ambient atmosphere. At the point where the through-going holes reaches the outer surface of the tubular member, the flow of the second part of the supplied gas is turned into a direction opposite the direction of the first flow direction.

The tubular member is provided with a shielding member, (e.g. sleeve or cuff), surrounding the air venting part for shielding the perforations and defining a space between the outer peripheral surface of the air venting part and the inner peripheral surface of the shielding member. The space may then be closed at a first end opposing the air outlet end part and open at an opposite second end part opposing the air inlet end. The shielding member thus in co-operation with the tubular member forms a flow channel for the air being released to the ambient atmosphere.

As an example, the shielding member may be a tubular, hollow body with an internal diameter or clearance, which exceeds the external diameter of the tubular member so that a circular flow passage is defined therein between. In one end, the cap seals against the outer surface of the tubular member towards the outlet end thereof. In the other end, the passage defined between the inner surface of the cap and the outer surface of the tubular member is open to the ambient atmosphere. Accordingly, gas entering through the through-going holes is guided through the circular flow passage from which passage it can only escape in a direction opposite the low pressure air outlet of the valve, i.e. in a direction facing away from the individual.

The shielding member may comprise a capsule-like member to be attached to the tubular member, the capsule having an internal peripheral portion with a flange section for shielding the perforation(s) and an attachment section for attaching it to the tubular member. The radial size of the flange section is larger than the radial size of the part of the tubular member comprising the perforation(s) whereas the radial size of the attachment section being equal to or smaller than the radial size of a part of the tubular member located between the perforation(s) and the air outlet.

Preferably, the radial sizes of the integrated unit and the attachment section of the shielding member, respectively, are adjusted so that the shielding member is fitted to the integrated unit by interference fitting and so that no air can escape through the fitting area towards the low pressure outlet. An O-ring may also be provided between the attachment section and the integrated unit, and/or there may be a groove and tongue connecting there between.

According to a preferred embodiment of the invention, the air inlet and outlet part, and the intermediate air venting part form an integrated unit, e.g. a one piece moulded tubular body of revolution. By having an integrated unit it provides that movable parts are avoided which could worsen the functionality of the valve. A particularly cost efficient valve may be made from two individually injection-moulded plastics or metallic parts, and wherein also the perforations in the venting part may be provided in the mould, e.g. by means of special designed cores in the mould.

A first of the parts may be an inner tubular body with a bore extending longitudinally between the high pressure air inlet and the low pressure air outlet. The tubular member may be provided with a number of through-going radially extending holes, i.e. holes extending perpendicularly to the first flow passage. A second of the parts may be an outer tubular cap. The shielding member is adapted in one end to seal against the outer surface of the tubular member whereas the other end is provided with a radial size allowing gas to escape between an inner surface of the cap and an outer surface of the tubular member, when the cap is attached to the tubular member.

The perforated area of the valve may extend throughout or substantially throughout the inner cavity of the valve, thus making the air venting part adapted to vent the major part of the air flowing through the inner cavity of the valve.

Moreover, the inner cavity and the perforation(s) of the air venting part could be shaped and dimensioned so as to reduce the air inlet pressure from an overpressure of several bars to an overpressure of a fraction of a bar. In particular, the dimensions of the high pressure inlet, the low pressure outlet, the air venting part and the perforations should be selected so that the air inlet pressure is reduced from the regular gas pressure supplies, i.e. normally 5-7 bar overpressure to the equivalence of 2-7 cm water column overpressure.

It may be an advantage to provide the shielding member as a separate part attached to the integrated unit in order to allow inspection of the venting part.

The integrated unit may form an external peripheral portion with a stepped configuration or with a flange, and the shielding member may then easily be positioned on the integrated unit by sliding the member onto the integrated unit until it engages a step or the flange of the peripheral portion of the integrated unit. In order to make the joint between the integrated unit and the shielding member (i.e. the attachment section of the shielding member) at least substantially airtight, the joined edges of at least one of the two parts may be provided with a surface with sealing characteristics. As an example, the inner surface of the attachment section of the shielding member part may thus be provided with a soft and resilient material, e.g. provided on a circumferentially and radially inwardly extending flange adapted to seal against the outer surface of the integrated unit. Alternatively, the outer surface of the integrated unit may be provided with such a material at least in the step or on the flange.

The flow of air from the inner cavity may provide an even better securing of the shielding member to the integrated unit, as the flow of air coming through the perforations provides a force (pressure) substantially perpendicular on the inner surface of the shielding member which thus provides a moment on the member so that the attachment section is pressed towards to outer surface of the integrated unit.

In order to support easy fitting of the valve for use in CPAP treatment or for treatment of sleep apnea or for similar treatment, the low pressure air outlet may be provided with a flange for connecting a nasal prong section. Alternatively, the low pressure air outlet may simply be formed as a set of nasal prongs allowing the air supply valve to be inserted directly into the nostrils.

During CPAP treatment of premature babies, the pressure reducing valve must be worn by the treated individual in relatively close vicinity to the respiratory organs. It may be positioned between 1-10 centimetres from the respiratory organs, preferably 3-4 centimetres from the respiratory organs. Accordingly, the weight of the valve is important for the well-being of the treated individual. Therefore, the weight may be between 0,5-10 grams, preferably 1,5 gram. Accordingly, the valve may preferably be made from a plastic material such as a thermoplast, which further provides that the patient can keep the valve on during scanning due to the translucent properties of plastic. In order to reduce the manufacturing costs, the valve may be made in one or two pieces by injection moulding.

The valve often needs to be positioned close to the baby in the couveuse (incubator), and thus the baby might get in touch with the valve. As it is very important that the valve delivers a constant pressure and flow of air the valve must be construed so that it is insensitive against external forces and other influences that could change the delivered flow of air by mistake. Therefore, the shielding member is preferably fixed attached to the tubular member so as to prevent the holes in the venting part to become obstructed, and thus secures a non-fluctuating pressure and flow of air.

Furthermore due to the fixed attachment of the shielding member, the valve can be used as a plug and play valve, thus an additional advantage is that it saves time for the personal working with the equipment.

The venting part may comprise one or a plurality of perforations. Preferably, the perforations of venting part comprise four air passages distributed around the circumference of the tubular member. The air pressure acting on the inner peripheral surface of the shielding member should preferably be evenly distributed in order to avoid pressure difference around the circumference thereof. Thus, the distribution of the perforations depends on the diameters of the perforations, so if the four passages have different diameter, they must be distributed unevenly around the circumference. Preferably, the passages are even distributed so that the angle between the centre lines of the air passages is substantially 90°, and the diameter of the one pair of oppositely arranged air passages may be different from the diameter of the other pair of oppositely arranged air passages. The diameter of the perforation(s) is preferably between 1-10 mm, such as 2-9 mm, such as 3-8 mm, such as 4-7 mm, such as 5-6 mm.

The diameter of the inlet end part may be between 4-8 mm, preferably 6 mm, and the diameter of the outlet end part is between 2-5 mm, preferably 3 mm.

The inner cavity and the perforation(s) of the air venting part may be shaped and dimensioned so as to reduce an air inlet overpressure of 6-7 bars to an air outlet overpressure of a few cm water column, such as 1-8 cm, such as 2-7 cm, such as 3-6 cm, such as 4-5 cm.

The distance between the outer surface of the air venting part and the inner surface of the shielding member is between 0.5-5 mm, preferably 1 or 2 mm. The air venting part is adapted to vent preferably 50% or more of the air flowing through the inner cavity of the valve into the ambient atmosphere.

The cross-section of the pressure reducing valve and the shielding member may have any shape, such as quadrangular or triangular or oval, but preferably they have a substantially circular cross-section.

Preferably the pressure-reducing valve according to the invention is disposable, and it may be pre-mounted in a “set” with an air supply tube connected to the high pressure inlet and maybe also with said nasal prong section connected to the low pressure outlet. The integrated unit may be moulded (injection moulded) together with the nasal prong section so as to provide one single moulded unit comprising the valve and the nasal prong. Preferably, the integrated unit and the nasal prong section is made of different type of material, respectively. The integrated unit may be made from unplasticised PVC (poly-vinyl-chloride) or polypropylene, while the nasal prong section may be made from soft PVC or silicone materials. When moulding the integrated unit together with the nasal prong section, it is preferred to have two mould-inlets each of the moulded parts.

According to a second aspect, the present invention relates to a method of providing gas to a CPAP valve, said method comprising conveying gas under pressure from a gas supply to at least two gas conveying passages, one passage extending towards an outlet in a first direction and the other passage extending towards an outlet in a direction oppositely in relation to the first direction.

The invention further relates to a method of providing gas to a CPAP (Continuous Positive Airway Pressure) valve, said method comprising conveying gas under a first pressure from a gas supply through a valve according to the first aspect of the invention.

According to a third aspect, the present invention relates to a tubular air supply device defining an inner cavity therein and comprising a high pressure air inlet end part, an opposite low pressure air outlet end part, an intermediate air venting part for venting air from the inner cavity into the ambient atmosphere and air flow deflecting means for directing air flows from the air venting part towards the air inlet end.

The air venting part may be a perforated wall part, and the perforated wall part may be an annular wall part.

The air flow deflecting means may comprise a sleeve or cuff surrounding the air venting part and defining a space between the outer peripheral surface of the air venting part and the inner peripheral surface of the sleeve or cuff, said space being closed at a first end opposing the air outlet end part and open at an opposite second end part opposing the air inlet end.

Preferably, the air inlet part, the air outlet part, and the intermediate air venting part form an integrated unit, and the integrated unit may be a tubular body of revolution.

The inner cavity and the air vents of the air venting part may be shaped and dimensioned so as to reduce the air inlet pressure from an overpressure of several bars to an overpressure of a fraction of a bar. The air venting part may be adapted to vent the major part of the air flowing through the inner cavity of the device.

The sleeve or cuff may be a separate part attached to the integrated unit.

The integrated unit may form an external peripheral portion intermediate the high pressure air inlet end part and the low pressure air outlet, said external peripheral portion comprising a stepped configuration or a flange adapted to position the sleeve or cuff.

The sleeve or cuff part may comprise an internal peripheral portion with a first section and a second section, the radial size of the first section being larger than the largest radial size of the integrated unit whereas the radial size of the second section being smaller than the radial size of the largest radial size of the integrated unit.

The low pressure air outlet may be adapted with a flange for connecting a nasal prong section, and the low pressure air outlet may be constituted by a nasal prong section having first and second nasal prong gas outlets. The sleeve or cuff part may be a tubular body of revolution.

It should be understood that any combination of the features and aspects mentioned is possible within the scope of the present application.

### DETAILED DESCRIPTION OF THE INVENTION

A preferred embodiment of the invention will now be described in details with reference to the drawings in which:

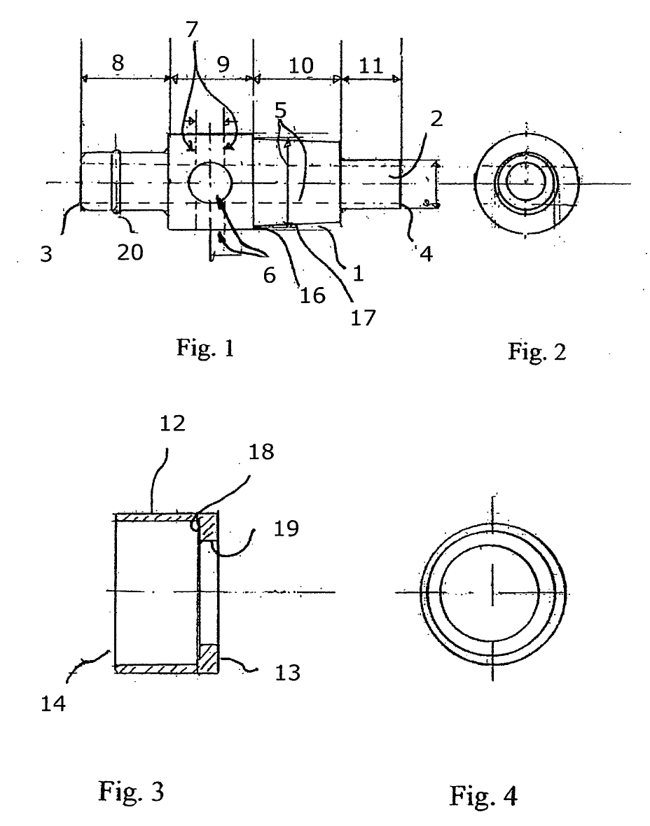

FIG. 1 shows a side view of a first part of a valve according to the present invention,

FIG. 2 shows the first part of FIG. 1, seen from the high pressure air inlet,

FIG. 3 shows a side view of a second part of a valve according to the present invention,

FIG. 4 shows the second part of FIG. 3, seen from an end thereof, which, when attached to the first part, is towards the high pressure air inlet,

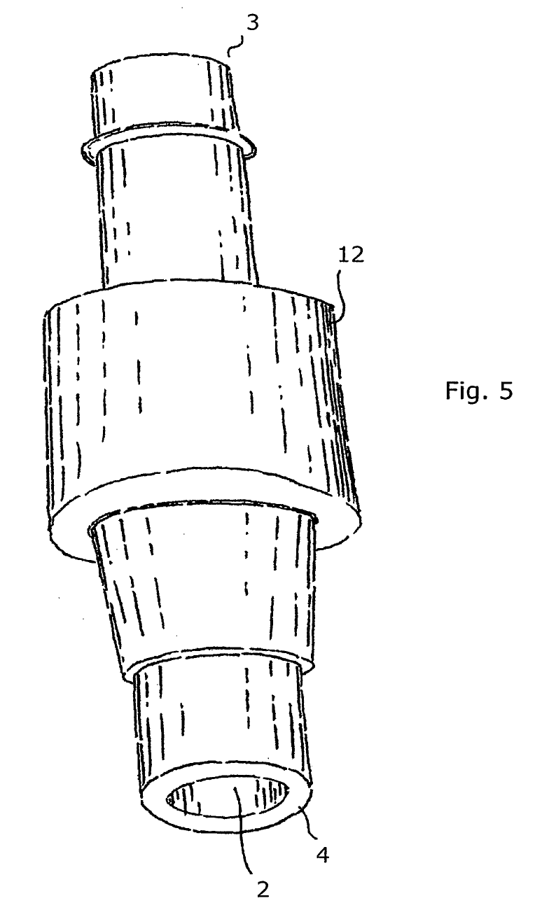

FIG. 5 shows a perspective view of a valve according to the present invention, and

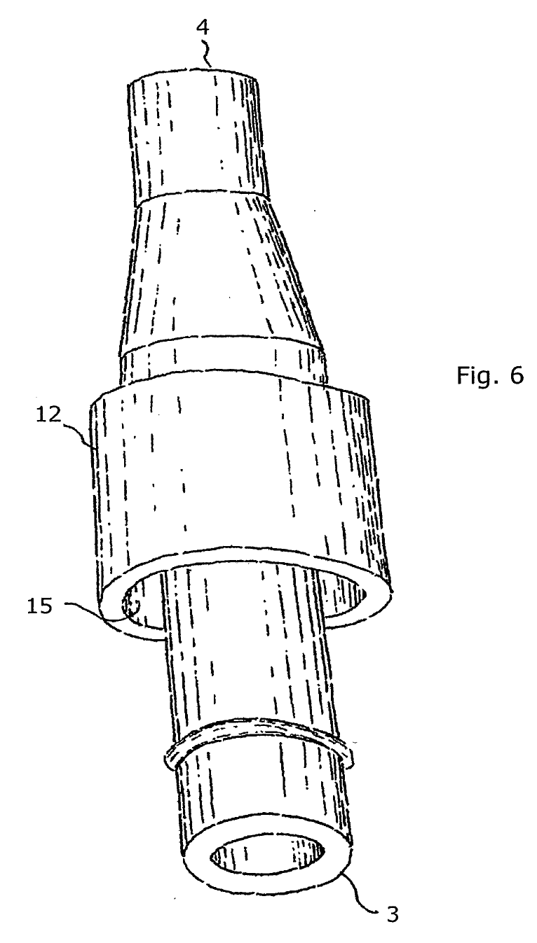

FIG. 6 shows another perspective view of the valve of FIG. 5.

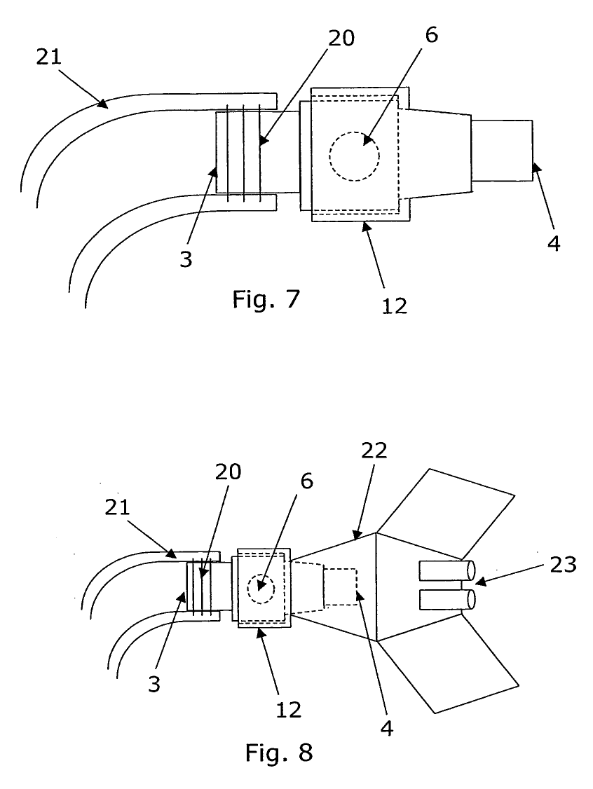

FIG. 7 shows a schematic view of the valve in combination with an air supply tube, and

FIG. 8 shows a schematic view of the valve in combination with both an air supply tube and a nasal prong section.

As shown in FIG. 1, a first part of a valve according to the present invention is an integrated unit comprising an oblong tubular member 1 provided with an internal flow passage 2. In FIG. 1, the passage is symbolised by the to dotted lines 5. The flow passage extends from a high pressure air inlet 3 to a low pressure air outlet 4. Intermediate between the inlet and the outlet, an air venting part is formed by perforation of the tubular member. In FIG. 1, the perforation is formed as four radially, throughout extending bore holes 6 allowing air to escape from the internal flow passage (inner cavity) to the ambient atmosphere (two of the bore holes is symbolised by the dotted lines 7).

As shown in FIG. 1, the oblong tubular member 1 may preferably have a stepped configuration with sections 8, 9, 10 and 11 having different external cross sectional sizes or diameters. Likewise, the flow passage may be split into sections having different cross-sectional sizes or diameters. At the high pressure inlet and/or at the low pressure air outlet, one or more resilient o-rings 20 may support air tight connection between the integrated unit and an air-supply hose and/or nasal prongs, respectively.

In FIG. 2, it is seen that the integrated unit is a tubular body of revolution.

In FIG. 3, the shielding member in the form of a second part 12, i.e. a sleeve or cuff part of the valve, is shown. The second part is provided with an elongated body having a pair of axially spaced, opposed ends 13,14. At one end 13, the second part is adapted to seal along an outer surface of the integrated unit part—an O-ring may be provided for sealing between said second part and the tubular member. At the other end 14 the second part in combination with the integrated unit, forms an outlet for the gas flowing through the air venting parts (perforations). The surface part 19 is adapted to slide against the surface part 17 of the integrated unit until the surface part 18 abuts against the surface part 16 of the integrated unit.

The stepped configuration of the external surface of the integrated unit will allow the second part to be positioned easily on to the integrated unit. As an example, one section of the first part may be provided with an external diameter allowing one section of the second part to be attached in a gas sealing engagement. Another section may be provided with a larger external diameter thus preventing the second part to slide over this section.

FIG. 5 shows a perspective view wherein the second part is attached to the first part.

FIG. 6 shows another perspective view wherein the passage defined between the integrated unit and the second part is shown. As shown, air entering through the high pressure air inlet 3 may flow in a substantially linear flow direction towards the low pressure air outlet 4, to which end nasal prongs or similar means for leading a flow of gas to the nostrils of a treated subject may be attached. Alternatively, nasal prongs may be an integrated part of the low pressure air outlet 4. Intermediate the two ends, a venting part may lead gas to the ambient atmosphere, which gas, by the second part 12 will be directed in a direction towards the high pressure gas inlet 3. In a preferred embodiment, the valve is provided with connecting flanges in one or both of the low and high pressure ends. The connecting flanges may e.g. be provided with one or more circumferentially extending protrusions, e.g. in the form of one or more O-shaped rubber rings. The high pressure end may be coloured in one colour and the low pressure end in another colour, thus allowing easy and safe orientation of the valve during the assembly with the gas supplies and the respiratory means. In FIG. 6, the flow channel 15 defined between the integrated unit or oblong body 1 and the second part 12, is clearly seen. The channel directs the air released via the air venting part from the inner cavity to the ambient atmosphere, in a direction opposite the direction of the low pressure air outlet.

FIG. 7 shows a schematic view of the valve in combination with an air supply tube 21. The air supply tube 21, connected to an air supplier in its inlet end, is connected to the high pressure inlet 3 of the valve. The connection of the tube is sealed by means of the moulded grooves and/or O-rings 20, but the connection may be sufficiently tight without said grooves or O-rings. The shielding member 12, the perforations 6 and the outlet 4 is also shown.

FIG. 8 shows a schematic view of the valve in combination with both an air supply tube 21 and a nasal prong section 22. The prong section is connected to the low pressure outlet end 4 of the valve. The prong section comprises two nasal prong air outlets 23 to be inserted into the nostrils. The valve and nasal prong section may be moulded together as one single unit.

1. A non-adjustable pressure reducing valve for nasal supplying of a flow of air to a patient, said valve comprising;
a hollowed tubular member defining an internal continuous flow passage extending from a high pressure air inlet end part, adapted to receive a flow of air from an air supplying conduit, to a low pressure air outlet end part opposite to said inlet end part and adapted to be connected to respiratory means for delivering a first part of said flow of air to the patient, and an intermediate air venting part having perforation(s) for venting a second part of said flow of air from said flow passage of the tubular member into the ambient atmosphere, and
a shielding member being attached to the tubular member such that a space is defined between an outer surface of the air venting part and an inner surface of the shielding member in order to provide a shield above said perforation(s), said space being closed at the end towards the air outlet end part and open at the opposite end towards the air inlet end for directing the second part of the air flow from said perforations towards said air inlet end part.

2. A valve according to claim 1, wherein the air inlet part, the air outlet part and the intermediate air venting part form an integrated unit.

3. A valve according to claim 1, wherein the valve is made in one piece.

4. A valve according to claim 1, wherein the shielding member comprises a capsule-like member to be attached to the tubular member, the capsule having an internal peripheral portion with a flange section for shielding said perforation(s) and an attachment section for attaching it to the tubular member, the radial size of the flange section being larger than the radial size of the part of the tubular member comprising the perforation(s) whereas the radial size of the attaching section being equal to or smaller than the radial size of a part of the tubular member located between the perforation(s) and the air outlet.

5. A valve according to claim 1, wherein the tubular member comprises an external peripheral portion between the high pressure air inlet end part and the low pressure air outlet, said external peripheral portion comprising a stepped configuration or a flange for receiving and holding said attachment section of the shielding member.

6. A valve according to claim 1, wherein the shielding member is a tubular body of revolution surrounding the air venting part.

7. A valve according to claim 1, wherein the shielding member is a separate part attached to the tubular member.

8. A valve according to claim 1, wherein the shielding member is fixed attached to the tubular member.

9. A valve according to claim 1, wherein the distance between said outer surface of the air venting part and said inner surface of the shielding member is between 0.5-5 mm, such as 1 or 2 mm.

10. A valve according to claim 1, wherein the diameter of the inlet end part is 4-8 mm, such as 6 mm.

11. A valve according to claim 1, wherein the diameter of the outlet end part is 2-5 mm, such as 3 mm.

12. A valve according to claim 1, wherein the perforations comprise four air passages substantially even distributed around the circumference of the tubular member.

13. A valve according to claim 12, wherein the angle between the centre lines of the air passages is substantially 90°.

14. A valve according to claim 13, wherein the diameter of the one pair of oppositely arranged air passages is different from the diameter of the other pair of oppositely arranged air passages.

15. A valve according to claim 1, wherein the diameter of the perforation(s) is between 1-10 mm, such as 2-9 mm, such as 3-8 mm, such as 4-7 mm, such as 5-6 mm.

16. A valve according to claim 1, wherein the valve is disposable.

17. A valve according to claim 1, wherein the inner cavity and the perforation(s) of the air venting part are shaped and dimensioned so as to reduce an air inlet overpressure of 6-7 bars to an air outlet overpressure of 2-7 cm water column.

18. A valve according to claim 1, wherein the air venting part is adapted to vent 50% or more of the air flowing through the inner cavity of the valve into the ambient atmosphere.

19. A valve according to claim 1, wherein the low pressure air outlet comprise a flange for connecting a nasal prong section thereto.

20. A valve according to claim 1, wherein the low pressure air outlet is constituted by a nasal prong section having first and second nasal prong air outlets.

21. A method of providing gas to a CPAP (Continuous Positive Airway Pressure) valve, said method comprising conveying gas under a first pressure from a gas supply to at least two gas passages, one passage extending towards an outlet in a direction towards a patient and the other passage extending towards an outlet in an opposite direction so as to reduce the pressure of the air coming out of the outlet to a pressure level below said first pressure.

22. A method of providing gas to a CPAP (Continuous Positive Airway Pressure) valve, said method comprising conveying gas under a first pressure from a gas supply through a valve comprising:
a hollowed tubular member defining an internal continuous flow passage extending from a high pressure air inlet end part, adapted to receive a flow of air from an air supplying conduit, to a low pressure air outlet end part opposite to said inlet end part and adapted to be connected to respiratory means for delivering a first part of said flow of air to the patient, and an intermediate air venting part having perforation(s) for venting a second part of said flow of air from said flow passage of the tubular member into the ambient atmosphere, and
a shielding member being attached to the tubular member such that a space is defined between an outer surface of the air venting part and an inner surface of the shielding member in order to provide a shield above said perforation(s), said space being closed at the end towards the air outlet end part and open at the opposite end towards the air inlet end for directing the second part of the air flow from said perforations towards said air inlet end part.
